(19) United States
(12) Patent Application Publication
(10) Pub. No.: US 2026/0021228 A1
(43) Pub. Date: Jan. 22, 2026

(54) PERITONEAL DIALYSIS CASSETTE WITH A BUBBLE DETECTOR

(71) Applicant: Mozarc Medical US LLC, Minneapolis, MN (US)

(72) Inventors: Gaspare Marotta, Mirandola (IT); Sunil Kumar Dayalan, Bangalore (IN); Francesca De Meis, Mirandola (IT); Rathnakara Narayana, Bangalore (IT)

(21) Appl. No.: 19/272,135

(22) Filed: Jul. 17, 2025

(30) Foreign Application Priority Data
Jul. 19, 2024 (IN) 202411055248

(51) Int. Cl. A61M 1/14; A61M 1/28; A61M 1/36

(52) U.S. Cl. CPC A61M 1/159; A61M 1/154; A61M 1/1561; A61M 1/28; A61M 1/3626; A61M 2205/3306

(57) ABSTRACT

Systems, components, and methods are provided for detection of air bubbles in a fluid moving through a peritoneal dialysis cassette. The systems, components and methods include a sensor capable of detecting air bubbles in the fluid positioned complementary to an air bubble sensing region of the peritoneal dialysis cassette.

### CROSS-REFERENCE TO RELATED APPLICATIONS

This application claims priority to Indian Patent Application number 202411055248, filed on Jul. 19, 2024 and titled “PERITONEAL DIALYSIS CASSETTE WITH A BUBBLE DETECTOR,” which is incorporated herein by reference in its entirety.

### FIELD

Systems, components, and methods are provided for detection of air bubbles in a fluid moving through a peritoneal dialysis cassette. The systems, components and methods include a sensor capable of detecting air bubbles in the fluid positioned complementary to an air bubble sensing region of the peritoneal dialysis cassette.

### BACKGROUND

Peritoneal dialysis is a form of renal replacement therapy whereby a catheter is placed in the peritoneal cavity and peritoneal dialysis fluid is introduced directly into the peritoneal cavity. Because peritoneal dialysis can be performed by a patient at home, easy to use and safe equipment for therapy is needed.

Air bubbles present in the fluid lines and cassettes of a peritoneal dialysis system can interfere with accurate measurement of peritoneal dialysis fluid and solutes, resulting in peritoneal dialysis fluid having incorrect composition, or peritoneal dialysis fluid in an incorrect volume. Hence, there is a need for systems and methods that can detect the presence of air bubbles in peritoneal dialysis fluid to ensure that the proper prescription for treatment is being delivered to the patient.

### SUMMARY OF THE INVENTION

The problem to be solved is detection of air bubbles in fluid moving through a peritoneal dialysis membrane. The solution is to use a sensor over a transparent portion of the peritoneal dialysis membrane to detect the presence of any air bubbles. In some embodiments, the sensor is an optical sensor. In some embodiments, the sensor is an ultrasound or ultrasonic sensor.

The first aspect relates to a peritoneal dialysis cassette. In some embodiments, the peritoneal dialysis cassette can include a first flexible surface and a second surface; a plurality of fluid passages fluidly connecting a plurality of inlet/outlet ports; the plurality of fluid passages between the first flexible surface and second e surface; a diaphragm pump having at least a first pump chamber and a second pump chamber; the diaphragm pump fluidly connected to the plurality of fluid passages; and at least one air bubble sensing region.

In some embodiments, the second surface can be a flexible surface; and the peritoneal dialysis cassette can have a rigid body between the first flexible surface and the second surface. In some embodiments, the second surface can be a rigid surface.

In some embodiments, the air bubble sensing region can have a transparent portion of the second rigid surface.

In some embodiments, the plurality of fluid passages can include closable fluid openings from a first side of the peritoneal dialysis cassette to a second side of the peritoneal dialysis cassette.

In some embodiments, the air bubble sensing region can have a membrane over a portion of the second surface.

In some embodiments, the air bubble sensing region can have a rigid cover over a portion of the second surface.

The features disclosed as being part of the first aspect can be in the first aspect, either alone or in combination, or follow any arrangement or permutation of any one or more of the described elements. Similarly, any features disclosed as being part of the first aspect can be in a second aspect described below, either alone or in combination, or follow any arrangement or permutation of any one or more of the described elements.

The second aspect relates to a system. In some embodiments, the system can include the peritoneal dialysis cassette of the first aspect; and a peritoneal dialysis cycler; the peritoneal dialysis cycler having an optical sensor; the optical sensor positioned complementary to the air bubble sensing region when the peritoneal dialysis cassette is engaged to the peritoneal dialysis cycler.

In some embodiments, the system can include a processor in communication with the optical sensor; the processor programmed to determine an amount of air bubbles present in the bubble sensing region.

In some embodiments, the processor can be programmed to drain fluid in the peritoneal dialysis cassette if air bubbles are present in the bubble sensing region.

In some embodiments, the second surface can be a rigid surface, and the air bubble sensing region can be a transparent portion of the first rigid surface.

In some embodiments, the air bubble sensing region can have a flat membrane over a portion of the second surface.

In some embodiments, the air bubble sensing region can have a transparent rigid cover over a portion of the second surface.

In some embodiments, the air bubble sensing region can be a transparent portion of the second flexible surface.

In some embodiments, the air bubble sensing region can have a flat membrane over a portion of the first flexible surface.

In some embodiments, the air bubble sensing region can have a transparent membrane over a portion of the first flexible surface.

In some embodiments, the plurality of fluid passages can include closable fluid openings from a first side of the peritoneal dialysis cassette to a second side of the peritoneal dialysis cassette.

In some embodiments, the system can include a processor; the processor programmed to operate one or more pinch valves to occlude or open the closable fluid openings.

A system can include a peritoneal dialysis cassette and a peritoneal dialysis cycler. The peritoneal dialysis cycler can include at least one ultrasonic sensor. The at least one ultrasonic sensor can be positioned complementary to the air bubble sensing region when the peritoneal dialysis cassette is engaged to the cycler.

In some embodiments, the at least one ultrasonic sensor is at least three ultrasonic sensors in series.

The features disclosed as being part of the second aspect can be in the second aspect, either alone or in combination, or follow any arrangement or permutation of any one or more of the described elements. Similarly, any features disclosed as being part of the second aspect can be in the first aspect, either alone or in combination, or follow any arrangement or permutation of any one or more of the described elements.

### BRIEF DESCRIPTION OF THE DRAWINGS

FIG. 1 shows a peritoneal dialysis cassette with a bubble sensing region, in accordance with some embodiments.

FIG. 2 shows a block diagram of an optical sensor system for detecting air bubbles in a peritoneal dialysis cassette, in accordance with some embodiments.

FIG. 3 shows a close-up view of a bubble sensing region in a peritoneal dialysis cassette, in accordance with some embodiments.

FIG. 4 shows a peritoneal dialysis cassette with a bubble sensing region, in accordance with some embodiments.

FIG. 5 shows a sensor, according to some embodiments.

FIG. 6A shows a cross section of a sensor, according to some embodiments.

FIG. 6B shows a cross section of a sensor, according to some embodiments.

### DETAILED DESCRIPTION

Unless defined otherwise, all technical and scientific terms used have the same meaning as commonly understood by one of ordinary skill in the art.

The articles “a” and “an” are used to refer to one to over one (i.e., to at least one) of the grammatical object of the article. For example, “an element” means one element or over one element.

An “air bubble sensing region” is a portion of a conduit, fluid passage, or cassette in which the presence of air bubbles can be detected.

The term “communication” refers to the ability to transmit or receive electronic information by any means known in the art.

The term “complementary,” when referring to the relative position of two or more components, refers to positions where the two components can interact as intended.

The term “comprising” includes, but is not limited to, whatever follows the word “comprising.” Use of the term indicates the listed elements are required or mandatory but that other elements are optional and may be present.

The terms “connected,” “connection,” to “connect,” or “connectable” refers to the ability of forming physical contact between two components or parts. The connection need not be permanent.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts, or features that do not affect the basic operation of the apparatus, structure or method described.

A “diaphragm pump” is a pump that operates to move fluid or gas by expanding and contracting a volume of one or more pump chambers.

To “drain” means to withdraw fluid from a component or system.

The term “engaged” refers to a physical connection formed between two components of a system.

The term “flat” refers to a material having a substantially planar surface.

A “flexible surface” refers to a surface of a component that can be deformed or bent without tearing or breaking.

A “fluid passage” is a conduit or pathway through which fluid or gas can move.

The term “fluidly connectable,” “fluid connection,” “fluidly connectable,” “fluidically engage,” or “fluidically coupled” refers to the ability of providing for the passage of fluid, gas, or combination thereof, from one point to another point. The ability of providing such passage can be any connection, fastening, or forming between two points to permit the flow of fluid, gas, or combinations thereof. The two points can be within or between any one or more of compartments of any type, modules, systems, components, and rechargers.

The term “fluidly connected” refers to a particular state such that the passage of fluid, gas, or combination thereof, is provided from one point to another point. The connection state can also include an unconnected state, such that the two points are disconnected from each other to discontinue flow. It will be further understood that the two “fluidly connectable” points, as defined above, can form a “fluidly connected” state. The two points can be within or between any one or more of compartments, modules, systems, components, and rechargers, all of any type.

An “inlet/outlet port” is an opening or conduit through which fluids can enter or exit a component.

A “membrane” is a thin material forming a boundary of a component.

The term “occlude” means to block a fluid passage, preventing fluid movement through the passage.

An “opening” is a conduit for a fluid or gas to move into, out of, or through a component.

An “optical sensor” is a sensor that detects optical properties of a fluid or material, such as light transmittance or reflectance of the material or fluid.

The term “peritoneal dialysis cassette” refers to a grouping of components that are arranged together for attachment to, or use with a peritoneal dialysis device, apparatus, or system. One or more components in a cassette can be any combination of single use, disposable, consumable, replaceable, or durable items or materials.

The term “peritoneal dialysis cycler” or “cycler” refers to components for movement of fluid into and out of the peritoneal cavity of a patient, with or without additional components for generating peritoneal dialysate or performing additional functions.

A “pinch clamp” is component that contacts a membrane surface to apply force to the membrane surface closing or opening a fluid conduit.

The term “processor” or “microprocessor” as used is a broad term and is to be given an ordinary and customary meaning to a person of ordinary skill in the art. The term refers without limitation to a computer system, state machine, processor, or the like designed to perform arithmetic or logic operations using logic circuitry that responds to and processes the basic instructions that drive a computer. In some embodiments, the terms can include ROM (“read-only memory”) and/or RAM (“random-access memory”) associated therewith.

The term “programmable” or “programmed” refers to an electronic system that can receive instructions to perform specified actions.

The terms “pumping” or to “pump” refer to the movement of a fluid or gas by application of pressure.

A “pump chamber” is an area into which fluid or a gas can flow having an expandable and contractable volume to draw in or expel the fluid or gas.

A “rigid cover” is a substantially inflexible material placed over a portion of a second component.

A “rigid surface” refers to a surface of a component that will generally not bend or deform during use.

The term “transparent” refers to a material through which light is able to pass.

### Disposable Peritoneal Dialysis Cassette

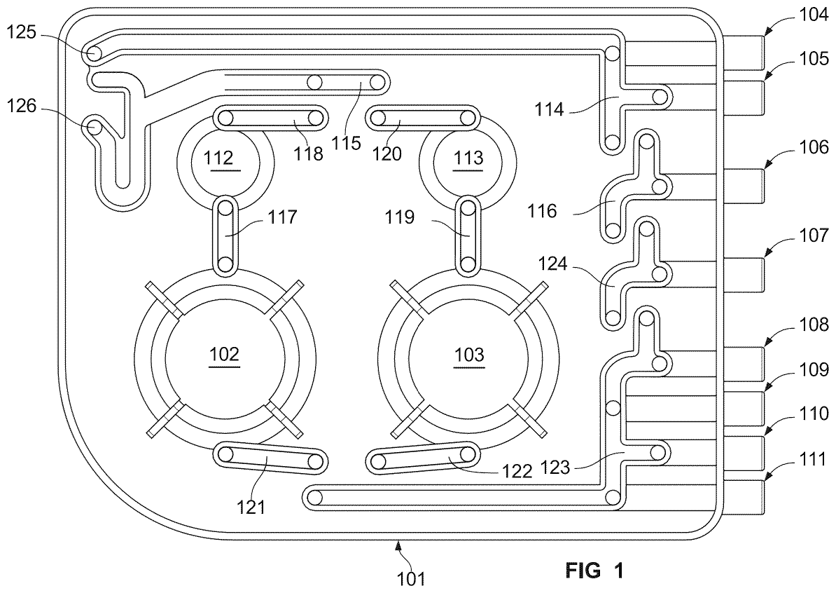

FIG. 1 shows a peritoneal dialysis cassette 101, according to some embodiments. FIG. 1 shows a bottom side of the peritoneal dialysis cassette 101. Although not shown in FIG. 1, the peritoneal dialysis cassette 101 has a first rigid surface on a top side and a second flexible surface on the bottom side.

In some embodiments, as shown in FIG. 1 the peritoneal dialysis cassette 101 can include one or more dedicated pressure sensing portions, illustrated as circular portion 112 and circular portion 113 for pressure monitoring. Circular portion 112 and circular portion 113 areas can work with two fluid contactless pressure sensors using the flexible surface on one side of the peritoneal dialysis cassette 101. The pressure sensors can detect the deformation of the flexible membrane surface due to fluid pressure in the peritoneal dialysis cassette 101.

The peritoneal dialysis cassette 101 can include one or more inlet/outlet ports.

Depending on the particular action being performed, fluid can be drawn into the peritoneal dialysis cassette 101 through any of the inlet/outlet ports and drawn out of the peritoneal dialysis cassette 101 through any of the inlet/outlet ports. That is, in some embodiments, each inlet/outlet port can be used as both an inlet and an outlet to perform specified actions.

In FIG. 1, the peritoneal dialysis cassette 101 includes a first inlet/outlet port 104 used for automatic sampling feature, a second inlet/outlet port 105 used for a drain line, a third inlet/outlet port 106 for the connection with a patient line, a fourth inlet/outlet port 107 used to connect to a heating bag line, a fifth inlet/outlet port 108 for a first pre-filled bag connection, a sixth inlet/outlet port 109 for a second pre-filled bag connection, a seventh inlet/outlet port 110 for a third pre-filled bag connection, and an eighth inlet/outlet port 111 for the connection with a fourth pre-filled bag. However, the peritoneal dialysis cassette can include any number of inlet/outlet ports, including more or fewer inlet/outlet ports than illustrated in FIG. 1. In some embodiments, the functions of each inlet/outlet port can be changed, adjusted, or swapped with the function of another inlet/outlet port. For example, in some embodiments, the order described above can be adjusted or changed.

As described, the peritoneal dialysis cassette can include a plurality of fluid passages. As illustrated in FIG. 1, inlet/outlet port 104 and inlet/outlet port 105 can be connected to fluid passage 114. Inlet outlet port 106 can be connected to fluid passage 116. Inlet/outlet port 107 can be connected to fluid passage 124. Inlet/outlet port 108, inlet/outlet port 109, inlet/outlet port 110, and inlet/outlet port 111 can each connect to fluid passage 123. Pump chamber 102 can be fluidly connected to fluid passage 117 and fluid passage 121, as well as fluid passage 118 through 112. A pump chamber 103 can be fluidly connected to fluid passage 119 and can be fluidly connected to fluid passage 122, as well as fluid passage 120 through 113. An additional fluid passage 115 can be included to facilitate the movement of fluid through the peritoneal dialysis cassette 101. One of skill in the art will understand that the number of fluid passages, as well as the arrangement of fluid passages can be varied. The design illustrated in FIG. 1 is for illustrative purposes only. In some embodiments, each fluid passage can be selectively occluded. By selectively occluding connections between the fluid passages, fluid can be moved through the peritoneal dialysis cassette 101 from any inlet/outlet port to any other inlet/outlet port.

An air bubble sensor (not shown in FIG. 1) can be included at any portion of the peritoneal dialysis cassette 101. As an example, an air bubble sensor can detect air bubbles on the top side of the peritoneal dialysis cassette 101 when fluid moves from the point labeled 126 to the point labeled 125. The portion of the fluid passage between points 125 and 126 can be the air bubble sensing region. The fluid, moving between points 125 and 126, can travel via the top side of the peritoneal dialysis cassette. In some embodiments, the sensor 201 can be an optical sensor configured to detect any air bubbles in the moving fluid. In some embodiments, the sensor 201 can be an ultrasonic sensor, a plurality of ultrasonic sensors, or any combination thereof, configured to detect air bubbles in the moving fluid. In some embodiments, other types of sensors can be used. For example, pressure, temperature, conductivity, flow rate, ultrasonic and/or other sensors can be used to detect air bubbles in the fluid, in some embodiments. Moreover, other positions can be included for air bubble detection. For example, in some embodiments, there can be air bubble sensors along or within any of the fluid passages described above. In some embodiments, the air bubble detection section can be placed as far as possible from any pinch clamps to avoid incorrect results due to deformation of the flexible membrane.

In some embodiments, the peritoneal dialysis cassette can include a pump, such as a diaphragm pump. The diaphragm pump can include a first pump chamber 102 and a second pumping chamber 103. When pressure is applied to the flexible membrane over first pump chamber 102, the flexible membrane is pushed inwardly towards the rigid side of the peritoneal dialysis cassette 101. As the flexible membrane is pushed inwardly, the fluid within the first pump chamber 102 can be acted upon and can be forced out of the first pump chamber 102. In some embodiments, the force from the membrane can be an increase in pressure. When the pressure on the flexible membrane over first pump chamber 102 can be released, the flexible membrane expands outwardly, drawing fluid into the first pump chamber 102. The same action forces fluid into and out of second pump chamber 103. As illustrated in FIG. 1, the first pump chamber 102 and the second pumping chamber 103 are fluidly connected to a plurality of fluid passages. Fluid moving through the peritoneal dialysis cassette 101 from one fluid passage to another fluid passage can pass through closable openings from a first side to a second side of the peritoneal dialysis cassette 101. Operating the diaphragm pump while selectively occluding passage of fluid through one or more of the closable openings allows fluid to be directed from a selected fluid inlet port to a selected fluid outlet port. In some embodiments, pinch clamps can be used as the valve system to selectively occlude the fluid openings and direct fluid through the peritoneal dialysis cassette 101. In some embodiments, the fluid passages can be occluded in other ways. For example, the fluid passages can be activatable to constrict, thereby blocking fluid flow. A processor of a control system (not shown) can be programmed to operate the pinch clamps, or other occlusion methods, to selectively direct fluid from a first specified inlet/outlet port to a second specified inlet/outlet port. As illustrated in FIG. 1, the first pump chamber 102 and second pumping chamber 103 can be symmetrical circular chambers. In some embodiments, other shapes can be used.

In some embodiments, other types of pumps can be included in the peritoneal dialysis cassette 101. For example, in some embodiments, an external pump can be used to drive fluid into and through the peritoneal dialysis cassette 101. In some embodiments, other internal pumps can also be used.

The peritoneal dialysis cassette 101 can be placed into a peritoneal dialysis cycler (not shown) for use. The peritoneal dialysis cycler can include components, such as a pneumatic system, for operating the diaphragm pump and pinch clamps of the peritoneal dialysis cassette. The peritoneal dialysis cycler can be connected to a patient for filling and/or draining of peritoneal dialysis fluid. In some embodiments, the dialysis cassette 101 can be used in a hemodialysis system.

Although shown as having one flexible membrane surface and one rigid surface in FIG. 1, the peritoneal dialysis cassette can, in some embodiments, have two flexible surfaces. Each flexible surface can cover one side of a rigid cassette body. Operation of a peritoneal dialysis system with a cassette having two flexible surfaces can proceed in the same manner as a cassette having a single flexible surface. Pinch clamps can be placed on either side of the rigid cassette body and press into the flexible surface on the same side to selectively occlude any specified fluid passage.

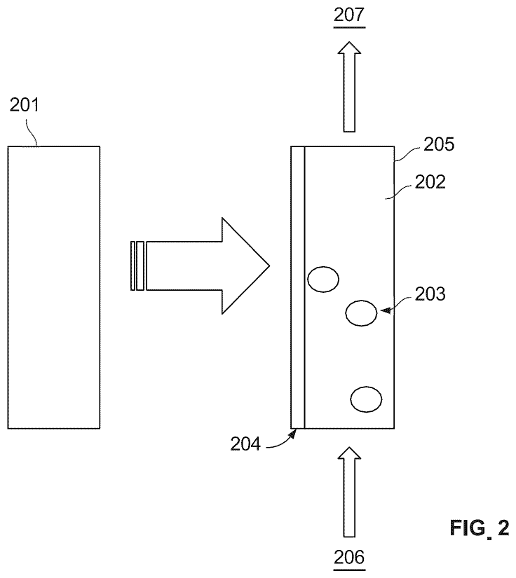

FIG. 2 is a block diagram showing the air bubble sensor. Fluid in a fluid passage 202 travels between a rigid surface 204 and a second rigid surface 205 in the fluid passage 202 in a direction of arrow 206 to arrow 207. In some embodiments, the sensor 201 can continuously or intermittently detect the properties such as optical properties of the fluid in fluid passage 202. In some embodiments, the sensor 201 can be an optical sensor. In some embodiments, the sensor 201 can be an ultrasonic sensor, a plurality of ultrasonic sensors, or any combination thereof, configured to detect air bubbles in the moving fluid. In some embodiments, other types of sensors can be used. For example, pressure, temperature, conductivity, flow rate, ultrasonic and/or other sensors can be used to detect air bubbles in the fluid, in some embodiments. As noted above, in some embodiments, any type of sensor capable of detecting air bubbles can be used. When an air bubble 203 passes in front of the sensor 201, the properties of the fluid will change. In some embodiments, the sensor 201 can be an optical sensor to detect changes in the optical properties of the fluid. In some embodiments not using an optical sensor, the fluid properties will change when an air bubble 203 passes through the fluid passage 202, which can be detected by the sensor. The sensor 201 can be in communication with a processor of a control system (not shown in FIG. 2) that is programmed to receive the data from the sensor and determine whether there are any air bubbles in the fluid. If any air bubbles are detected, the control system can stop the process or issue an alert or alarm to let the user know that air bubbles are present in the fluid. In some embodiments, the processor can be programmed to automatically stop the process or to drain all fluid from the peritoneal dialysis cassette 101 in response to air bubbles being present in the fluid. The processor can also be programmed to determine the amount or volume of air bubbles in the fluid.

As illustrated in FIG. 2, the sensor 201 can be placed on the rigid surface side of the peritoneal dialysis cassette 101. A translucent or transparent window can be built into the rigid surface to allow the sensor 201 to detect air bubbles in the fluid. In some embodiments, a portion of the rigid surface over the air bubble sensing region can be replaced with a translucent or transparent flat membrane to aid in the detection of air bubbles. In other embodiments, the sensor 201 can be built into, or be integral with, either surface of the peritoneal dialysis cassette.

The sensor 201 can, in some embodiments, operate by detecting deviation in light due to the presence of bubbles in the fluid. In some embodiments, the optical sensor can detect reflected light. Air bubbles in the fluid stream can change the amount of light reflected from the fluid stream. Deviations in the detected reflected light can be used to determine the difference between a continuative fluid stream and a fluid stream having air bubbles. In some embodiments, optical sensors that can measure other optical properties of the fluid can also be used.

In other embodiments the sensor 201 can be placed on the flexible membrane side (not shown in FIG. 2) of the peritoneal dialysis cassette. The flexible membrane can be translucent or transparent to aid the sensor 201 in detecting air bubbles in the fluid. In some embodiments, an opaque flexible membrane can be used and a translucent or transparent window can be constructed in the flexible membrane to aid the detection of air bubbles.

The sensor 201 can, in some embodiments, be affixed or built into the peritoneal dialysis cycler (not shown in FIG. 2). In some embodiments, the position of the sensor 201 can be fixed such that, when the peritoneal dialysis cassette is properly engaged with the cycler, the optical sensor is positioned complementary to the air bubble sensing region, permitting the detection of air bubbles in the fluid passage 202 of the peritoneal dialysis cassette. In some embodiments, the position of the sensor 201 can be adjustable, depending on the desired use. For example, in some situations, the user can place the sensor 201 to detect air bubbles in a specific fluid passage. This can, in some embodiments, permit more control of the detection of air bubbles within the cassette 101.

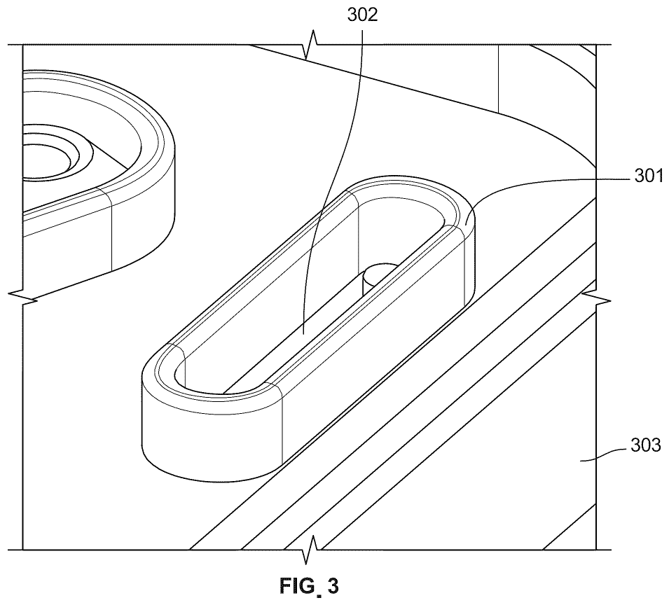

FIG. 3 shows a close-up view of a bubble sensing region 301 in a peritoneal dialysis cassette 303. FIG. 3 shows the bubble sensing region 301 on the top side of the peritoneal dialysis cassette 303, however, as described, the bubble sensing region can instead be located on the bottom side of the peritoneal dialysis cassette 303. As described, fluid can move through a fluid passage 302 from the bottom side of the peritoneal dialysis cassette 303 through openings from the bottom to the top side of the peritoneal dialysis cassette. When the fluid is in fluid passage 302, and sensor 201 (not shown in FIG. 3) can be positioned above the air bubble sensing region 301, the sensor 201 can determine if any bubbles are passing through the bubble sensing region 301. If so, the system can provide an alert to the user or stop any ongoing process.

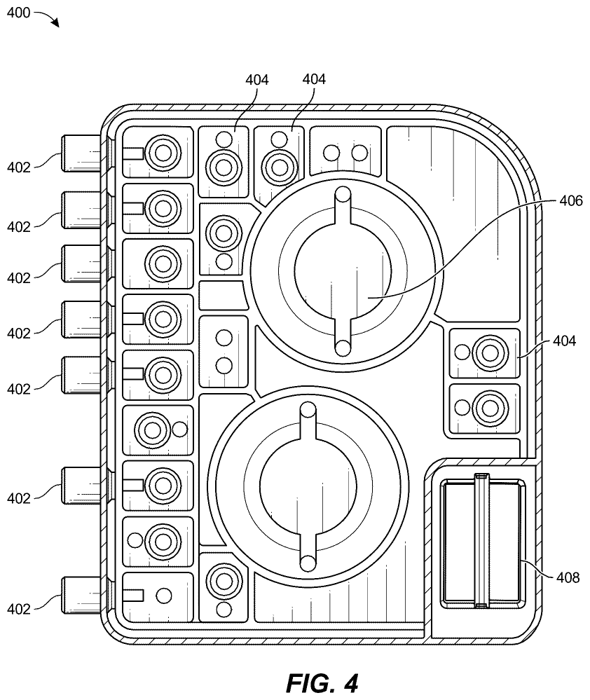

Turning to FIG. 4, an embodiment of an peritoneal dialysis cassette 400 is shown. Similar to the peritoneal dialysis cassette 101 shown in FIG. 1, peritoneal dialysis cassette 400 can have a plurality of ports 402 through which fluid can enter the peritoneal dialysis cassette 400 and exit the peritoneal dialysis cassette 400. In some embodiments, the plurality of ports 402 can serve as fluid ingress and egress into and out of the peritoneal dialysis cassette 400.

In some embodiments, the peritoneal dialysis cassette 400 can include a plurality of valves 404 that connect various fluid passages, as shown in FIG. 1, throughout the peritoneal dialysis cassette 400. The plurality of valves 404 can be selectively opened and closed to deliver fluid to, or from, a desired port of the plurality of ports 402.

In some embodiments, the peritoneal dialysis cassette 400 can include at least one pump chamber 406 to pump fluid through the fluid passages disposed through the peritoneal dialysis cassette 400, shown in FIG. 1. In some embodiments, the at least one pump chamber 406 can be pneumatically activated by a cycler (not shown) interfacing with the peritoneal dialysis cassette 400.

In some embodiments, the peritoneal dialysis cassette 400 can include a sensor 408 to detect the presence of gas within a fluid through the sensor 408. The sensor 408 can be in communication with a processor of a control system that is programmed to receive the data from the sensor and determine whether there are any air bubbles in the fluid. If any air bubbles are detected, the control system can stop the current process, issue an alert to let the user know that air bubbles are present in the fluid, sound an alarm to let the user know that air bubbles are present in the fluid, or any combination thereof.

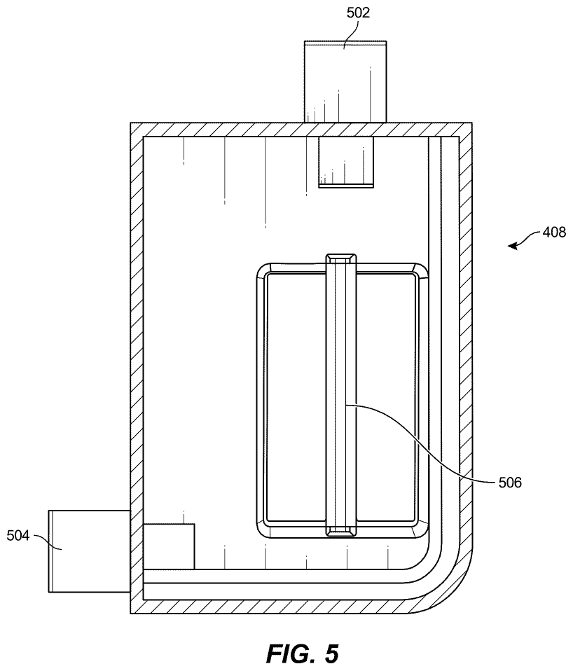

In reference to FIG. 5, the sensor 408 is shown, according to some embodiments. The sensor 408 can include a first port 502 and a second port 504. In some embodiments, fluid can be delivered to the sensor 408 through the first port 502. The fluid in the sensor 408 can then travel through a sensor fluid passageway 506. In some embodiments, the sensor fluid passageway 506 can be fluidly connected to the first port 502 and the second port 504. After traveling through the sensor fluid passageway 506, the fluid can exit the sensor 408 through the second port 504. In some embodiments, fluid can be delivered to the sensor 408 through the second port 504. The fluid in the sensor 408 can then travel through a sensor fluid passageway 506. In some embodiments, the sensor fluid passageway 506 can be fluidly connected to the first port 502 and the second port 504. After traveling through the sensor fluid passageway 506, the fluid can exit the sensor 408 through the first port 502. In some embodiments, the sensor 408 can include only port. For example, fluid can enter the sensor 408, the sensor 408 can measure fluid properties of the fluid disposed therein. Once data has been collected, the sensor 408 can output that data to a control system and the fluid can be removed from the sensor 408 through the same entry port, e.g., either the first port 502 or the second port 504.

In some embodiments, the sensor fluid passageway 506 can be a portion of the sensor 408 through which, while fluid is flowing through the sensor fluid passageway 506, the sensor 408 is collecting fluid data about the fluid disposed in the sensor fluid passageway 506. For example, while fluid is flowing through the sensor fluid passageway 506, the sensor 408 can collect fluid data used by a control system to determine the presence of air, gas, or any combination thereof, disposed within the fluid traveling through the sensor fluid passageway 506.

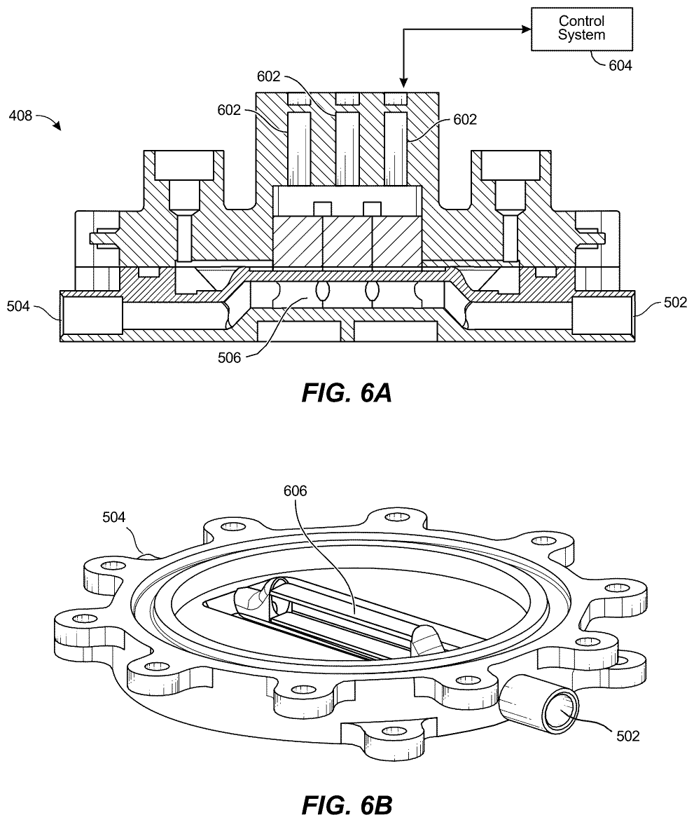

In reference to FIG. 6A and FIG. 6B the sensor 408 can include a plurality of sensors 602. In some embodiments, the plurality of sensors 602 can be capacitance sensors. In some embodiments, the plurality of sensors 602 can be ultrasound sensors. For example, as fluid travels through the sensor fluid passageway 506, the plurality of sensors 602 can send ultrasonic waves through the fluid in the sensor fluid passageway 506. The air volume can then, in some embodiments, be estimated by a control system 604 based on the time the ultrasonic waves require to travel through the sensor fluid passageway 506, including the fluid disposed therein. For example, the control system 604 can determine there is no gas within the fluid in the sensor fluid passageway 506 based on the measurements from the plurality of sensors 602. In some embodiments, the presence of a gas within the fluid disposed within the sensor fluid passageway 506 will affect the speed through which the ultrasonic waves travel through the sensor fluid passageway 506, which can be measured by the plurality of sensors 602.

The sensor fluid passageway 506 can, in some embodiments, be designed to minimize abrupt discontinuities which may affect the measurements of the plurality of sensors 602. For example, the sensor fluid passageway 506 can have all rounded edges, substantially all rounded edges, a majority of rounded edges, or any combination thereof, to minimize the affect of geometry on the plurality of sensors 602.

In some embodiments, the sensor fluid passageway 506 can include a sensing region 606. The sensing region 606 can be a region of the sensor fluid passageway 506 that interfaces with the plurality of sensors 602. In some embodiments, the sensing region 606 can be a region of the sensor fluid passageway 506 that receives the ultrasonic waves from the plurality of sensors 602. In some embodiments, the sensing region 606 can be shaped to minimize refraction of the ultrasonic waves from the plurality of sensors 602. For example, in some embodiments, the sensing region 606 can have a consistent cross-section. By having a consistent cross-section, abrupt discontinuities that may affect the sensor measurements from sensor 408 can be minimized, leading to better measurements from the sensor 408.

In some embodiments, the control system 604 can communicate with the sensor 408 to receive the fluid properties measured by the sensor 408. The control system 604 can use the measurements from the sensor 408 to determine whether there is a gas, air, bubbles, or any combination thereof, disposed within the fluid. If the control system 604 determines that there is gas, air, bubbles, or any combination thereof, disposed within the fluid, the control system 604 can adjust the operation of the peritoneal dialysis cassette 400 based on that determination. For example, in some embodiments, the control system 604 can shut down operation of the peritoneal dialysis cassette 400. In some embodiments, the control system 604 can direct fluid to a specific portion of the peritoneal dialysis cassette 400 that can be configured for air removal.

In some embodiments, the plurality of sensors 602 can include at least three individual ultrasound sensors. In some embodiments, the plurality of sensors 602 can include at least three individual ultrasound sensors, in series. In some embodiments, the plurality of sensors 602 can include at least two individual ultrasound sensors. In some embodiments, the sensor 408 can include only a single ultrasound sensor.

One skilled in the art will understand that various combinations and/or modifications and variations can be made in the described systems and methods depending upon the specific needs for operation. Various aspects disclosed herein may be combined in different combinations than the combinations specifically presented in the description and accompanying drawings. Moreover, features illustrated or described as being part of an aspect of the disclosure may be used in the aspect of the disclosure, either alone or in combination, or follow a preferred arrangement of one or more of the described elements. Depending on the example, certain acts or events of any of the processes or methods described herein may be performed in a different sequence, may be added, merged, or left out altogether (e.g., certain described acts or events may not be necessary to carry out the techniques). In addition, while certain aspects of this disclosure are described as performed by a single module or unit for purposes of clarity, the techniques of this disclosure may be performed by a combination of units or modules associated with, for example, a medical device.

What is claimed is:

1. A peritoneal dialysis cassette; comprising:
a first flexible surface and a second surface;
a plurality of fluid passages fluidly connecting a plurality of inlet/outlet ports, the plurality of fluid passages being disposed between the first flexible surface and the second surface;
a diaphragm pump comprising a first pump chamber and a second pump chamber, the diaphragm pump fluidly connected to the plurality of fluid passages; and
at least one air bubble sensing region.

2. The peritoneal dialysis cassette of claim 1, wherein the second surface is a flexible surface; the peritoneal dialysis cassette further comprising a rigid body between the first flexible surface and the second surface.

3. The peritoneal dialysis cassette of claim 1, wherein the second surface is a rigid surface.

4. The peritoneal dialysis cassette of claim 3; wherein the air bubble sensing region comprises a transparent portion of the second surface.

5. The peritoneal dialysis cassette of claim 1; wherein the plurality of fluid passages include closable fluid openings from a first side of the peritoneal dialysis cassette to a second side of the peritoneal dialysis cassette.

6. The peritoneal dialysis cassette of claim 3, wherein the air bubble sensing region comprises a membrane over a portion of the second surface.

7. The peritoneal dialysis cassette of claim 3, wherein the air bubble sensing region comprises a rigid cover over a portion of the second surface.

8. A system, comprising:
the peritoneal dialysis cassette of claim 1;
a peritoneal dialysis cycler;
the peritoneal dialysis cycler comprising an optical sensor;
the optical sensor positioned complementary to the air bubble sensing region when the peritoneal dialysis cassette is engaged to the cycler.

9. The system of claim 8, further comprising a processor in communication with the optical sensor; the processor programmed to determine an amount of air bubbles present in the bubble sensing region.

10. The system of claim 9, the processor programmed to drain fluid in the peritoneal dialysis cassette if air bubbles are present in the bubble sensing region.

11. The system of claim 8, wherein the second surface is a rigid surface, and wherein the air bubble sensing region is a transparent portion of the second surface.

12. The system of claim 11, wherein the bubble sensing region comprises a flat membrane over a portion of the second surface.

13. The system of claim 11, wherein the air bubble sensing region comprises a transparent rigid cover over a portion of the second surface.

14. The system of claim 8, wherein the air bubble sensing region is a transparent portion of the first flexible surface.

15. The system of claim 14, wherein the air bubble sensing region comprises a flat membrane over a portion of the first flexible surface.

16. The system of claim 14, wherein the air bubble sensing region comprises a transparent membrane over a portion of the first flexible surface.

17. The system of claim 8, wherein the plurality of fluid passages include closable fluid openings from a first side of the peritoneal dialysis cassette to a second side of the peritoneal dialysis cassette.

18. The system of claim 17, further comprising a processor; the processor programmed to operate one or more pinch clamps to occlude or open the closable fluid openings.

19. A system, comprising:
the peritoneal dialysis cassette of claim 1;
a peritoneal dialysis cycler; the peritoneal dialysis cycler comprising at least one ultrasonic sensor;
wherein the at least one ultrasonic sensor is positioned complementary to the air bubble sensing region when the peritoneal dialysis cassette is engaged to the cycler.

20. The system of claim 19, wherein the at least one ultrasonic sensor is at least three ultrasonic sensors in series.
